(19) United States
(12) Patent Application Publication
(10) Pub. No.: US 2026/0096819 A1
(43) Pub. Date: Apr. 9, 2026

(54) Absorbable Anastomotic Protection Device and Fecal Diversion Device for Post-Colorectal Surgery

(71) Applicants: Jing Lu, Rancho Cucamonga, CA (US); Yong Zhang, Xi'an (CN); Jie Shao, Xi'an (CN); Zicai Zhu, Xi'an (CN)

(72) Inventors: Jing Lu, Rancho Cucamonga, CA (US); Yong Zhang, Xi'an (CN); Jie Shao, Xi'an (CN); Zicai Zhu, Xi'an (CN)

(21) Appl. No.: 18/906,174

(22) Filed: Oct. 4, 2024

(51) Int. Cl. A61B 17/12; A61B 17/00; A61M 3/02

(52) U.S. Cl. CPC A61B 17/1204; A61B 17/12136; A61M 3/0295; A61B 2017/00004; A61B 2017/00818; A61B 2217/007

(57) ABSTRACT

This application relates to a colorectal surgical device, specifically an absorbable anastomotic protection and fecal diversion device for post-colorectal surgery. It includes a fecal diversion device and an absorbable elastic band. The fecal diversion device comprises an inflatable flushing section, two balloon air tubes, a cleaning tube, and an intestinal protective film. The protective film has a hollow conduit housing the tubes. Two supporting balloons are spaced apart and sleeved on the distal end of the protective film, with the absorbable elastic band positioned between them. Made of absorbable polymer material, the device provides temporary anastomosis protection and fecal diversion after colorectal surgery. The external part naturally absorbs post-healing, while the internal device is easily removable.

### BACKGROUND OF THE INVENTION

This invention relates to the field of colorectal surgical instruments, particularly to an absorbable device for postoperative anastomosis protection and fecal diversion in colorectal surgery.

Postoperative colorectal anastomosis is susceptible to contamination from intestinal contents, leading to infection, slow healing, or serious complications like anastomotic leakage, especially after low rectal anastomosis. Preventing anastomotic infection primarily involves the prophylactic diversion of residual feces from the intestine to avoid contact with the anastomosis site.

Current technologies typically use simple supporting anal tubes or intestinal decompression tubes for drainage. For example, patent CN216061445U discloses an intestinal decompression tube for postoperative anal decompression and anastomotic leakage in mid-to-low rectal surgery, which discharges intestinal contents through an intermediate balloon and drainage tube. However, this device lacks external intestinal tightening mechanisms, leading to tube slippage and poor sealing between the decompression tube and the intestinal wall, risking residual feces contaminating the anastomosis site.

Thus, there is an urgent clinical need for a high-sealing, easily removable anastomosis protection and fecal diversion device.

### SUMMARY OF THE INVENTION

This application discloses an absorbable device designed for anastomosis protection and fecal diversion following colorectal surgery. The device serves to temporarily safeguard the anastomotic site and divert fecal matter post-surgery. Following tissue healing, the external tightening mechanism naturally absorbs, facilitating easy removal of the internal diversion device.

To address the technical issues mentioned above, the application introduces an absorbable device for anastomosis protection and fecal diversion post-colorectal surgery. This device comprises a fecal diversion apparatus and an absorbable elastic band used to secure the apparatus to the external intestinal wall. The fecal diversion apparatus includes an inflatable irrigation section, a first balloon catheter, a cleansing tube, a second balloon catheter, an intestinal protective membrane, a first support balloon, and a second support balloon. The intestinal protective membrane has a hollow passage; one end of the first balloon catheter, the cleansing tube, and the second balloon catheter are located within this hollow passage, while the other end extends outside the intestinal protective membrane and is connected to the opening of the inflatable irrigation section. The first and second support balloons are positioned at the end of the intestinal protective membrane away from the inflatable irrigation section. The first support balloon is connected to the first balloon catheter, and the second support balloon is connected to the second balloon catheter. The absorbable elastic band is positioned between the first and second support balloons and is composed of an absorbable polymer.

In some exemplary embodiments, one end of the absorbable elastic band is provided with a tightening fixing buckle, and the other end is provided with several adjustment fixing holes that match the tightening fixing buckle.

In some exemplary embodiments, the absorbable elastic band is located between the first support balloon and the second support balloon; during use, the absorbable elastic band is fixed into a ring shape.

In some exemplary embodiments, the first support balloon and the second support balloon are tightened and constrained to the outer intestinal wall by the absorbable elastic band, ensuring that the first support balloon and the second support balloon are tightly sealed against the inner intestinal wall.

In some exemplary embodiments, the material of the absorbable elastic band is an absorbable polymer composed of polyglycolide and polylactide.

In some exemplary embodiments, the openings of the inflation and flushing part include a first balloon port, a second balloon port, and a cleaning port.

In some exemplary embodiments, the first balloon port is connected to the first balloon air tube, the second balloon port is connected to the second balloon air tube, and the cleaning port is connected to the cleaning tube, with one-way valves provided at the connections of the first balloon port with the first balloon air tube, the second balloon port with the second balloon air tube, and the cleaning port with the cleaning tube.

In some exemplary embodiments, the side wall of the intestinal protective film is provided with a cleaning hole, which is connected to the cleaning tube.

In some exemplary embodiments, the cleaning hole is located where the intestinal protective film contacts the anastomotic site.

In some exemplary embodiments, during use, the fecal diversion device is inserted into the intestinal cavity through the anus, with part of the surface of the intestinal protective film located outside the intestinal cavity.

The technical solution provided by this embodiment of the present application at least has the following advantages:

This embodiment of the present application provides an absorbable anastomotic site protection and fecal diversion device for post-operative colorectal surgery, comprising: a fecal diversion device and an absorbable elastic band for tightening the fecal diversion device to the outer intestinal wall; the fecal diversion device includes an inflation and flushing part, a first balloon air tube, a cleaning tube, a second balloon air tube, an intestinal protective film, a first support balloon, and a second support balloon. The intestinal protective film has a hollow duct, with one end of the first balloon air tube, the cleaning tube, and the second balloon air tube located inside the hollow duct of the intestinal protective film, and the other end located outside the intestinal protective film and the intestinal cavity, and connected to the openings of the inflation and flushing part; the first support balloon and the second support balloon are spaced apart and sleeved at the end of the intestinal protective film far from the inflation and flushing part, with the first support balloon connected to the first balloon air tube, and the second support balloon connected to the second balloon air tube; the absorbable elastic band is located between the first support balloon and the second support balloon; the absorbable elastic band is made of an absorbable polymer.

The absorbable anastomotic site protection and fecal diversion device provided by this embodiment of the present application adds an absorbable tightening device for the outer intestinal wall, i.e., an absorbable elastic band, ensuring that the internal balloons are tightly sealed against the intestinal inner wall. After providing protection for a period of time, the elastic band gradually degrades and is completely absorbed, facilitating the removal of the fecal diversion device without causing perforation injuries to the body. Additionally, a cleaning hole is added to the side wall of the intestinal protective film of the fecal diversion device, allowing for flushing and disinfection of the anastomotic site and the surrounding intestinal cavity as needed.

### BRIEF DESCRIPTION OF DRAWINGS

The embodiments of the device are exemplified in the accompanying figures, which illustrate without limitation:

FIG. 1 is a structural diagram of the device in use.

FIG. 2 is a structural diagram of the fecal diversion apparatus.

FIG. 3 is a structural diagram of the absorbable elastic band.

FIG. 4 is an annular structure diagram of the tightened absorbable elastic band.

Reference numerals: 1—Intestinal cavity, 2—Fecal diversion apparatus, 3—Absorbable elastic band, 11—Anastomotic site, 21—Inflatable irrigation section, 22—First balloon catheter, 23—Cleansing tube, 24—Second balloon catheter, 25—Intestinal protective membrane, 26—First support balloon, 27—Second support balloon, 31—Fastening buckle, 32—Adjustment holes.

### DETAILED DESCRIPTION OF THE INVENTION

From the background, it is known that existing anastomotic protection and fecal diversion devices lack an external intestinal fastening mechanism at the front end. Consequently, the decompression tube tends to slip, and the sealing between the decompression tube and the intestinal wall is often poor, resulting in residual feces leaking and contaminating the anastomosis.

To address the above technical issues, this embodiment provides an absorbable anastomotic protection and fecal diversion device for post-colorectal surgery. The device comprises a fecal diversion device and an absorbable elastic band for fastening the fecal diversion device to the external intestinal wall. The fecal diversion device includes an inflation rinsing section, a first balloon airway, a cleaning tube, a second balloon airway, an intestinal protective membrane, a first supporting balloon, and a second supporting balloon. The intestinal protective membrane has a hollow conduit where one end of the first balloon airway, cleaning tube, and second balloon airway is positioned. The other end of these components is located outside the intestinal protective membrane and intestinal cavity and is connected to the opening of the inflation rinsing section. The first supporting balloon and the second supporting balloon are spaced apart and sleeved at the end of the intestinal protective membrane distant from the inflation rinsing section. The first supporting balloon is connected to the first balloon airway, and the second supporting balloon is connected to the second balloon airway. The absorbable elastic band is situated between the first supporting balloon and the second supporting balloon and is made of absorbable polymer material. The absorbable anastomotic protection and fecal diversion device provided by this embodiment can be used for temporary protection of the anastomosis and fecal diversion after colorectal surgery. The external fastening mechanism naturally absorbs after healing, facilitating the removal of the internal diversion device.

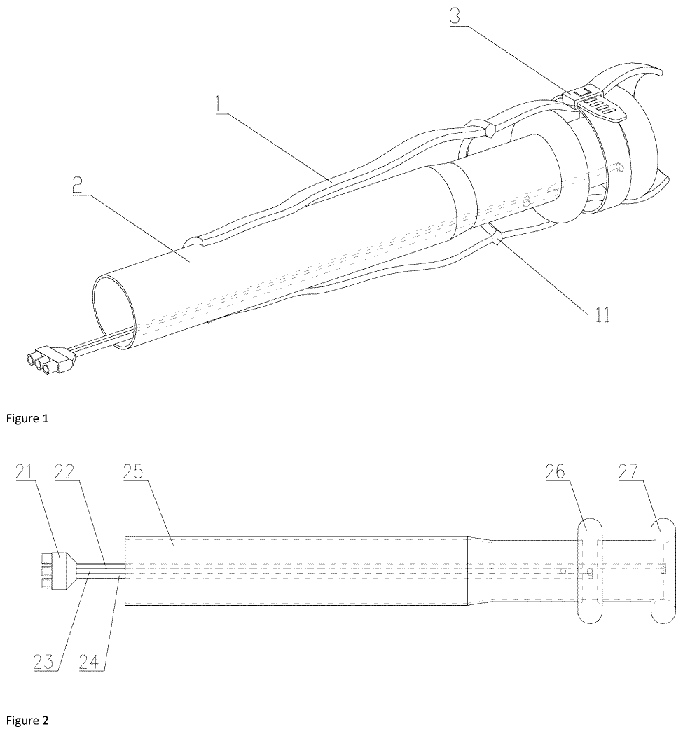

Referring to FIG. 1 and FIG. 2, the absorbable anastomotic protection and fecal diversion device for post-colorectal surgery includes a fecal diversion device (2) and an absorbable elastic band (3) for fastening the fecal diversion device (2) to the external intestinal wall. The fecal diversion device (2) comprises an inflation rinsing section (21), a first balloon airway (22), a cleaning tube (23), a second balloon airway (24), an intestinal protective membrane (25), a first supporting balloon (26), and a second supporting balloon (27). The intestinal protective membrane (25) has a hollow conduit where one end of the first balloon airway (22), cleaning tube (23), and second balloon airway (24) is positioned. The other end of these components is located outside the intestinal protective membrane (25) and is connected to the opening of the inflation rinsing section (21). The first supporting balloon (26) and the second supporting balloon (27) are spaced apart and sleeved at the end of the intestinal protective membrane (25) distant from the inflation rinsing section (21). The first supporting balloon (26) is connected to the first balloon airway (22), and the second supporting balloon (27) is connected to the second balloon airway (24). The absorbable elastic band (3) is situated between the first supporting balloon (26) and the second supporting balloon (27), and is made of absorbable polymer material.

The following will provide a detailed explanation of the various embodiments of the present application in conjunction with the accompanying drawings. However, ordinary technicians in this field can understand that many technical details have been proposed in various embodiments of the present application to help readers better understand the present application. However, even without these technical details and various changes and modifications based on the following embodiments, the technical solution claimed in this application can still be achieved.

The absorbable anastomotic protection and fecal diversion device provided by this application can be used for temporary protection of the anastomosis after colorectal surgery. During use, the fecal diversion device (2) is inserted into the intestinal cavity (1) through the anus. The supporting balloons, consisting of the first balloon airway (22) and the second balloon airway (24), are inflated and tensioned once positioned at the front of the anastomosis (11). The central sections of the two balloons are fastened to the external intestinal wall by the absorbable elastic band (3), ensuring a tight seal with the internal wall. The central sections of the balloons have through holes, allowing intestinal contents such as feces to be expelled through the hole. Additionally, the fecal diversion device (2) includes cleaning holes and associated cleaning tubes at the anastomosis (11) location, enabling necessary rinsing and disinfection of the anastomosis (11) and surrounding intestinal cavity. After approximately two weeks, the anastomosis (11) heals, the tension of the absorbable elastic band (3) gradually fails and is fully absorbed, and the deflated fecal diversion device (2) can be removed. This application offers temporary protection for the intestinal anastomosis, reducing the probability of anastomotic leakage and facilitating removal, thus having high practical value.

As shown in FIG. 1, a portion of the fecal diversion device (2) is located outside the intestinal cavity (1), while the other part is inside the intestinal cavity (1). Specifically, as shown in FIG. 2, the inflation rinsing section (21) and one end of the intestinal protective membrane (25) near the inflation rinsing section (21) are outside the intestinal cavity (1), while the first supporting balloon (26), the second supporting balloon (27), and the absorbable elastic band (3) are inside the intestinal cavity (1).

Please continue to refer to FIG. 2. The intestinal protective membrane (25) has a hollow conduit, and the first balloon airway (22), cleaning tube (23), and second balloon airway (24) are positioned inside the hollow conduit of the intestinal protective membrane (25). The intestinal protective membrane (25) includes a first conduit and a second conduit, with the outer diameter of the first conduit being larger than that of the second conduit, and the first and second conduits being connected through a connecting section. The first conduit is closer to the inflation rinsing section (21) compared to the second conduit, with part of the first conduit being outside the intestinal cavity (1) and the other part of the first conduit and the second conduit being inside the intestinal cavity (1). The central sections of the first supporting balloon (26) and the second supporting balloon (27) have a through-hole structure. The first supporting balloon (26) and the second supporting balloon (27) are sleeved on the second conduit, with the second supporting balloon (27) located at the end of the second conduit away from the first conduit. The absorbable elastic band (3) is situated between the first supporting balloon (26) and the second supporting balloon (27), used to fasten the first supporting balloon (26) and the second supporting balloon (27) to the external intestinal wall.

In some embodiments, during use, the fecal diversion device (2) is inserted into the intestinal cavity through the anus, with part of the surface of the intestinal protective membrane (25) positioned outside the intestinal cavity.

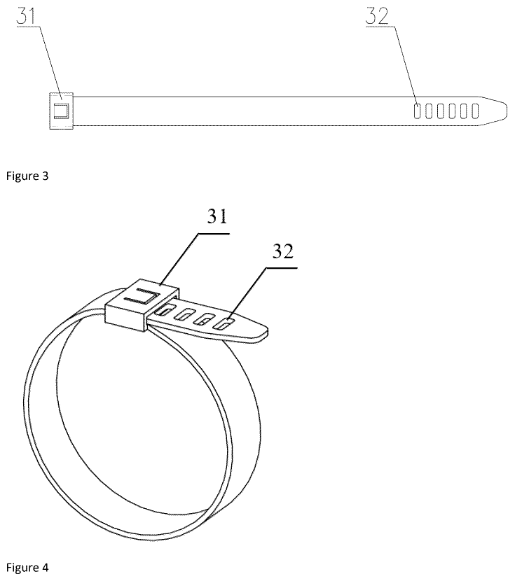

In some embodiments, as shown in FIG. 3, one end of the absorbable elastic band (3) is provided with a tightening fixing buckle (31), and the other end is provided with several adjustment fixing holes (32) matching the tightening fixing buckle (31).

In some embodiments, the absorbable elastic band (3) is positioned between the first supporting balloon (26) and the second supporting balloon (27); as shown in FIG. 4, the absorbable elastic band (3) is fixed in a loop during use.

In some embodiments, the first supporting balloon (26) and the second supporting balloon (27) are fastened to the external intestinal wall by the absorbable elastic band (3), ensuring a tight seal between the first supporting balloon (26) and the second supporting balloon (27) and the internal wall.

In some embodiments, the absorbable elastic band (3) is made of an absorbable polymer composed of glycolide and lactide. The absorbable elastic band (3) has a certain elasticity, with a support time of about two weeks, gradually losing tension thereafter, and being fully absorbed within one to two months.

In some embodiments, the opening of the inflation rinsing section (21) includes a first balloon port, a second balloon port, and a cleaning port.

As shown in FIG. 2, in some embodiments, the first balloon port is connected to the first balloon airway (22), the second balloon port is connected to the second balloon airway (24), and the cleaning port is connected to the cleaning tube (23). One-way valves are provided at the connections between the first balloon port and the first balloon airway (22), the second balloon port and the second balloon airway (24), and the cleaning port and the cleaning tube (23). The one-way valves can be opened during inflation rinsing and closed after use to ensure the sealing of the first supporting balloon (26) and the second supporting balloon (27). The balloons can be manually deflated when removal is required.

In some embodiments, the sidewall of the intestinal protective membrane (25) is provided with cleaning holes connected to the cleaning tube (23).

In some embodiments, the cleaning holes are positioned at the contact location between the intestinal protective membrane (25) and the anastomosis (11).

The method of using the device of the present invention is as follows:
1. During use, the fecal diversion device (2) is inserted into the intestinal cavity through the anus. After placing the two support balloons (the first support balloon (26) and the second support balloon (27)) in front of the anastomosis (11), they are inflated and tightened. The middle of the two support balloons is constrained by an absorbable elastic band (3) to tighten the external wall of the intestine, ensuring that the balloons are closely sealed against the inner wall. The middle of the balloon is a through hole, allowing the discharge of fecal and other digestive contents from the intestinal cavity through the hole. Simultaneously, the fecal diversion device is provided with a cleaning hole at the anastomosis position and a cleaning tube connected to the cleaning hole, which can flush and disinfect the anastomosis and surrounding intestinal cavity as needed;
2. After approximately two weeks, the anastomosis (11) heals, the tension of the absorbable elastic band (3) gradually fails, and it is then completely absorbed. The fecal diversion device's balloons are deflated and removed.

According to the above technical solution, the embodiment of the present application provides an absorbable device for anastomotic protection and fecal diversion after colorectal surgery, comprising: a fecal diversion device (2) and an absorbable elastic band (3) for tightening the fecal diversion device (2) to the external wall of the intestine; the fecal diversion device (2) includes an inflation and irrigation part (21), a first balloon tube (22), a cleaning tube (23), a second balloon tube (24), an intestinal protective film (25), a first support balloon (26), and a second support balloon (27); wherein the intestinal protective film (25) has a hollow duct, one end of the first balloon tube (22), the cleaning tube (23), and the second balloon tube (24) is located inside the hollow duct of the intestinal protective film (25), and the other end is located outside the intestinal protective film (25) and the intestinal cavity, and the other end is connected to the opening of the inflation and irrigation part (21); the first support balloon (26) and the second support balloon (27) are sleeved at intervals on the end of the intestinal protective film (25) away from the inflation and irrigation part (21), and the first support balloon (26) is connected to the first balloon tube (22), and the second support balloon (27) is connected to the second balloon tube (24); the absorbable elastic band (3) is located between the first support balloon (26) and the second support balloon (27); the material of the absorbable elastic band (3) is an absorbable polymer.

The embodiment of the present application provides an absorbable device for anastomotic protection and fecal diversion after colorectal surgery, which ensures the tight sealing of the internal balloons against the intestinal wall by adding an absorbable external wall tightening device, namely, the absorbable elastic band (3), to the fecal diversion device (2). After providing temporary protection, the absorbable elastic band (3) gradually fails and is completely absorbed, facilitating the removal of the fecal diversion device (2) without causing perforation injury to the body. Additionally, a cleaning hole is added to the sidewall of the intestinal protective film (25) of the fecal diversion device (2), allowing for the flushing and disinfection of the anastomosis and surrounding intestinal cavity as needed.

It should be understood by those skilled in the art that the above embodiments are specific examples for implementing the present application. Various modifications and changes can be made in form and detail without departing from the spirit and scope of the present application. Therefore, the protection scope of the present application should be determined by the appended claims.

1. The absorbable anastomotic protection and fecal diversion device for post-colorectal surgery, comprising: A fecal diversion device and an absorbable elastic band for securing the fecal diversion device to the outer wall of the intestine. The fecal diversion device includes an inflatable flushing section, a first balloon air tube, a cleaning tube, a second balloon air tube, an intestinal protective film, a first supporting balloon, and a second supporting balloon; wherein the intestinal protective film has a hollow conduit, with one end of the first balloon air tube, the cleaning tube, and the second balloon air tube located inside the hollow conduit of the intestinal protective film, and the other end located outside the intestinal protective film and intestinal lumen, connected to the opening of the inflatable flushing section. The first supporting balloon and the second supporting balloon are spaced apart and sleeved on the end of the intestinal protective film away from the inflatable flushing section, with the first supporting balloon connected to the first balloon air tube and the second supporting balloon connected to the second balloon air tube. The absorbable elastic band is located between the first supporting balloon and the second supporting balloon, made of an absorbable polymer.

2. The absorbable anastomotic protection and fecal diversion device according to claim 1, characterized in that: One end of the absorbable elastic band is provided with a fastening buckle, and the other end is provided with several adjustment holes matching the fastening buckle.

3. The absorbable anastomotic protection and fecal diversion device according to claim 1, characterized in that: The absorbable elastic band is located in the middle position between the first supporting balloon and the second supporting balloon. During use, the absorbable elastic band is fixed in a ring shape.

4. The absorbable anastomotic protection and fecal diversion device according to claim 1, characterized in that: The first supporting balloon and the second supporting balloon are tightened and constrained by the absorbable elastic band against the outer wall of the intestine, ensuring the first supporting balloon and the second supporting balloon are in close sealing contact with the inner wall of the intestine.

5. The absorbable anastomotic protection and fecal diversion device according to claim 1, characterized in that: The absorbable elastic band is made of an absorbable polymer composed of glycolide and lactide.

6. The absorbable anastomotic protection and fecal diversion device according to claim 1, characterized in that: The opening of the inflatable flushing section includes a first balloon port, a second balloon port, and a cleaning port.

7. The absorbable anastomotic protection and fecal diversion device according to claim 6, characterized in that: The first balloon port is connected to the first balloon air tube, the second balloon port is connected to the second balloon air tube, and the cleaning port is connected to the cleaning tube, with check valves provided at the connections of the first balloon port and the first balloon air tube, the second balloon port and the second balloon air tube, and the cleaning port and the cleaning tube.

8. The absorbable anastomotic protection and fecal diversion device according to claim 1, characterized in that: Cleaning holes are provided on the side wall of the intestinal protective film, and the cleaning holes are connected to the cleaning tube.

9. The absorbable anastomotic protection and fecal diversion device according to claim 8, characterized in that: The cleaning holes are located at the position where the intestinal protective film contacts the anastomotic site.

10. The absorbable anastomotic protection and fecal diversion device according to claim 1, characterized in that: During use, the fecal diversion device is inserted into the intestinal lumen through the anus, with part of the surface of the intestinal protective film located outside the intestinal lumen.
